(19) United States
(12) Patent Application Publication
(10) Pub. No.: US 2008/0207533 A1
(43) Pub. Date: Aug. 28, 2008

(54) Pharmaceutical Compositions

(76) Inventors: Katja Jores, Binningen (CH); Andreas Meyer, Neuenburg (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/909,312

(22) PCT Filed: Mar. 23, 2006
(86) PCT No.: PCT/EP2006/002693

§ 371 Date: Sep. 21, 2007

Related U.S. Application Data
(60) Provisional application No. 60/664,733, filed on Mar. 24, 2005.

(51) Int. Cl. A61K 31/7068

(52) U.S. Cl. 514/25

(57) ABSTRACT

A pharmaceutical composition and granulates, prepared by a wet granulation process. The pharmaceutical composition and granulates contain a therapeutic compound, e.g., the 3′-L-valine ester of β-D-2′-C-methyl ribofuranosyl cytidine and its salts, esters, prodrugs or derivatives.

### FIELD OF THE INVENTION

The present invention relates to pharmaceutical compositions comprising a therapeutic compound. Furthermore, the present invention relates to a wet granulation process for preparing a granulate containing the therapeutic compound that may be used to prepare a pharmaceutical composition.

### BACKGROUND OF THE INVENTION

The Flaviviridae family of viruses causes a variety of diseases in mammals, e.g., dengue fever, yellow fever and hepatitis C in humans; bovine viral diarrhea in cattle, border disease in sheep; and swine fever in pigs. Of particular concern, for humans, is hepatitis C which is caused by the hepatitis C virus (“HCV”) which is the leading cause of chronic liver disease worldwide. HCV causes a slow growing viral infection and is the major cause of cirrhosis and hepatocellular carcinoma. An estimated 170 million persons are infected with HCV worldwide. Cirrhosis caused by chronic hepatitis C infection accounts for 8,000 to 12,000 deaths per year in the United States, and HCV infection is the leading indication for liver transplantation.

The 3′-L-valine ester of β-D-2′-C-methyl-ribofuranosyl cytidine (“val-mCyd”) is an effective therapeutic compound useful for the treatment of flaviviridae, especially HCV. The compound val-mCyd or its salts, esters, prodrugs or derivatives are described in PCT Publication No. WO 2004/002422 (published on Jan. 8, 2004) which is hereby incorporated by reference in its entirety. This patent publication describes the nature and use of val-mCyd or its salts, esters, prodrugs or derivatives as being anti-Flaviviridae agents.

Particularly useful forms of val-mCyd, e.g., include its salts, such as β-D-2′-C-methyl-ribofuranosyl cytidine-3′-O-L-valine ester-HCl and the β-D-2′-C-methyl-ribofuranosyl cytidine dihydrochloride salt (“Compound I”). The structures for val-mCyd and Compound I are shown below:

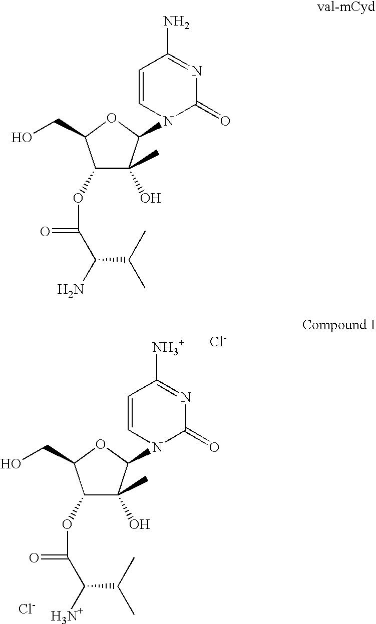

An object of the present invention is to provide a novel granulate or a pharmaceutical composition, e.g., a solid oral dosage form, containing val-mCyd and/or its salts, esters, prodrugs or derivatives thereof. A further object of the present invention is to provide a wet granulation process for making such a novel granulate that may be used in such solid oral dosage forms.

### SUMMARY OF THE INVENTION

The present invention provides for a novel granulate containing a therapeutic compound, such as val-mCyd or its salts, esters, prodrugs and derivatives. These granulates may be further processed such that they are incorporated into a solid oral dosage form. Particularly useful as a process for preparing granulates is wet granulation.

In one exemplary embodiment of the present invention, the wet granulation process to prepare granulates includes the following steps:
a) forming a powder blend of the therapeutic compound, e.g., val-mCyd and at least one pharmaceutically acceptable excipient;
b) adding a granulation liquid to the powder blend under agitation to form a wet mass;
c) granulating the wet mass to form moist granulates; and
d) drying the moist granulates.

In another exemplary embodiment of the present invention, the granulates prepared by the exemplary wet granulation process are further incorporated into a solid oral dosage form, e.g., a tablet. This process includes the following steps:
a) forming a powder blend of the therapeutic compound, e.g., val-mCyd and at least one pharmaceutically acceptable excipient;
b) adding a granulation liquid to the powder blend under agitation to form a wet mass;
c) granulating the wet mass to form moist granulates;
d) drying the moist granulates to form granulates;
e) sieving the granulates;
f) blending the sieved granulates with additional pharmaceutically acceptable excipients to form a mixture; and
g) compressing the mixture to form a tablet.

In yet another exemplary embodiment of the present invention, the wet granulation prepared granulates are incorporated into another type of solid oral dosage form, e.g., a capsule. The granulates can also be used as multiparticulates for other solid oral dosage forms. This process includes the following steps:
a) forming a powder blend of the therapeutic compound, e.g., val-mCyd and at least one pharmaceutically acceptable excipient;
b) adding a granulation liquid to the powder blend under agitation to form a wet mass;
c) granulating the wet mass to form moist granulates;
d) drying the moist granulates to form granulates; and
e) encapsulating the granulates in a capsule.

Alternatively, the granulates may be blended with other pharmaceutically acceptable excipients and encapsulated to form a capsule if so desired.

### DETAILED DESCRIPTION OF THE INVENTION

The present invention relates to a novel process for preparing, especially through wet granulation, granulates of val-mCyd and at least one pharmaceutically acceptable excipient. The present invention also relates to solid oral dosage forms prepared from such granulates.

As used herein, the term “val-mCyd” refers to the 3′-L-valine ester of β-D-2′-C-methyl-ribofuranosyl cytidine.

As used herein, the terms “salt” and “ester” refer to any pharmaceutically acceptable form (e.g., an ester, phosphate ester, salt of an ester or a related group) of a nucleoside compound which upon administration to a patient, provides the nucleoside compound.

Pharmaceutically acceptable salts include those derived from pharmaceutically acceptable inorganic or organic bases and acids. Suitable salts include those derived from alkali metals, e.g., potassium and sodium, alkaline earth metals, such as calcium and magnesium, among numerous other acids well-known in the pharmaceutical art. Examples of salts include, but are not limited to, hydrochloride tosylate, methanesulfonate, acetate, mesylate, citrate, malonate, tartarate, succinate, benzoate, ascorbate, α-ketoglutarate, α-glycerophosphate, formate, fumarate, propionate, glycolate, lactate, pyruvate, oxalate, maleate, salicylate, sulfate, sulfonate, nitrate, bicarbonate, hydrobromate, hydrobromide, carbonate and phosphoric acid salts. Particularly useful salts of val-mCyd include the monohydrochloride and dihydrochloride salts of val-mCyd.

Pharmaceutically acceptable prodrugs refer to a compound that is metabolized, e.g., hydrolyzed or oxidized, in the patient to form the nucleoside compound. Examples of prodrugs include compounds that have biologically labile protecting groups on a functional moiety of the active compound. Prodrugs include drugs that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. The compounds of the present invention possess anti-viral activity against a Flaviviridae, or are metabolized to a compound that exhibits such activity.

As used herein, the term “therapeutic compound” collectively refers to val-mCyd and/or its salts, esters, prodrugs or derivatives, e.g., Compound I.

As used herein, the term “pharmaceutical composition” means, e.g., a mixture or solution containing a therapeutically effective amount of a therapeutic compound in a pharmaceutically acceptable carrier to be administered to a mammal, e.g., a human in order to prevent, treat or control flaviviridae (including HCV) infections and other related conditions, such as anti-HCV antibody positive and HCV-positive conditions, and hepatitis C related cancer (e.g., hepatocellular carcinoma) and hepatic tumors. Moreover, these pharmaceutical compositions may be used prophylactically to prevent or retard the progression of clinical illness in individuals who are anti-HCV (or more generally anti-flaviviridae) antibody or HCV-antigen or flaviviridae-antigen positive, or who have been exposed to HCV or another flaviviridae virus.

As used herein, the term “pharmaceutically acceptable” refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

The therapeutic compound is present in the pharmaceutical compositions of the present invention in a therapeutically effective amount or concentration. Such a therapeutically effective amount or concentration is known to one of ordinary skill in the art. For example, the dose of the therapeutic compound will be in the range from about 1-50 mg/kg, e.g., 1-20 mg/kg, of body weight per day, more generally 0.1 mg/kg to about 100 mg/kg body weight of the recipient per day. Alternatively lower doses may be given, e.g., doses of 0.5-100 mg/kg, 0.5-50 mg/kg, 0.5-10 mg/kg or 0.5-5 mg/kg body weight per day. Furthermore, even lower doses may be useful, and thus ranges can include from 0.1-0.5 mg/kg body weight per day. The effective dosage range of the pharmaceutically acceptable salts and prodrugs can be calculated based on the weight of the parent nucleoside to be delivered. If the salt or prodrug exhibits activity itself, the effective dosage can be estimated as above using the weight of the salt or prodrug, or by other means known to those skilled in the art.

The compound is conveniently administered in unit any suitable dosage form including, but not limited to, one containing 7-3,000 mg, e.g., 70-1,400 mg of therapeutic compound per unit dosage form. An oral dosage of 50-1,000 mg is usually convenient, including in one or multiple dosage forms of 50, 100, 200, 250, 300, 400, 500, 600, 700, 800, 900 or 1000 mg. Alternatively, lower doses may be administered, e.g., from 10-100 mg or 1-50 mg. Also contemplated are doses of 0.1-50 mg, 0.1-20 mg or 0.1-10 mg.

The therapeutic compound can be administered to achieve peak plasma concentrations of the therapeutic compound of from about 0.2-70 μM, e.g., about 0.1-10.0 μM.

The concentration of the therapeutic compound in the pharmaceutical composition will depend on absorption, inactivation and excretion rates of the drug, as well as other factors known to one of ordinary skill in the art. Furthermore, it is to be noted that dosage values will also vary with the severity of the condition to be alleviated. It is to be further understood that for any particular recipient, specific dosage regimens should be adjusted over time according to the individual need and the professional judgment of the person administering or supervising the administration of the pharmaceutical compositions. The therapeutic compound can be administered once, or can be divided into a number of smaller doses to be administered at varying intervals of time.

As used herein, the term “excipient” refers to a pharmaceutically acceptable ingredient that is commonly used in the pharmaceutical technology for preparing granulate and/or solid oral dosage formulations. Examples of categories of excipients include, but are not limited to, binders, disintegrants, lubricants, glidants, stabilizers, fillers and diluents. One of ordinary skill in the art may select one or more of the aforementioned excipients with respect to the particular desired properties of the granulate and/or solid oral dosage form by routine experimentation and without any undue burden. The amount of each excipient used may vary within ranges conventional in the art. The following references which are all hereby incorporated by reference discloses techniques and excipients used to formulate oral dosage forms. See The Handbook of Pharmaceutical Excipients, 4^{th} edition, Rowe et al., Eds., American Pharmaceuticals Association (2003); and Remington: the Science and Practice of Pharmacy, 20^{th} edition, Gennaro, Ed., Lippincott Williams & Wilkins (2000).

As used herein, the term “immediate-release” refers to the rapid release of the majority of the therapeutic compound, e.g., greater than about 50%, about 55%, about 60%, about 65%, about 70%, about 75%, about 80% or about 90% within a relatively short time, e.g., within 1 hour, 40 minutes, 30 minutes or 20 minutes after oral ingestion. Particularly useful conditions for immediate-release are release of at least or equal to about 80% of the therapeutic compound within 30 minutes after oral ingestion. The particular immediate-release conditions for a specific therapeutic compound will be recognized or known by one of ordinary skill in the art.

As used herein, the term “wet granulation” refers to the general process of using a granulation liquid in the granulation process to subsequently form granulates, as discussed in Remington: The Science and Practice of Pharmacy, 20^{th} Edition (2000), Chapter 45, which is hereby incorporated by reference.

In an exemplary embodiment of the present invention, wet granulation includes the steps of mixing; wetting and kneading, i.e., wet massing; granulating; drying; and sieving. These steps are discussed in more detail below.

The wet granulation process begins with the formation of a powder blend of the therapeutic compound and at least one pharmaceutically acceptable excipient by mixing with, e.g., pharmaceutical granulation equipment, the aforementioned ingredients (i.e. bringing into intimate proximity) in a suitable container, so as to form a mixture. Examples of pharmaceutical granulation equipment include but are not limited to, shear granulators (e.g., Hobart, Collette, Beken) in combination with an oscillating granulator; high-speed mixers/granulators (e.g., Diosna, Fielder, Collette-Gral); and fluidized-bed granulators (e.g., Aeromatic, Glatt) with a subsequent sieving equipment. Excipients useful for initially mixing with the therapeutic compound include, e.g., binders, fillers, disintegrants, diluents and any combinations of the foregoing.

Examples of pharmaceutically acceptable disintegrants include, but are not limited to, starches; clays; celluloses; alginates; gums; cross-linked polymers, e.g., cross-linked polyvinyl pyrrolidone or crospovidone, e.g., POLYPLASDONE XL from International Specialty Products (Wayne, N.J.); cross-linked sodium carboxymethylcellulose or croscarmellose sodium, e.g., AC-DI-SOL from FMC; and cross-linked calcium carboxymethylcellulose; soy polysaccharides; and guar gum. The disintegrant, e.g., may be present in an amount from about 1% to about 20%, e.g., from about 5% to about 10%, e.g., about 5% by weight of the composition.

Examples of pharmaceutically acceptable binders include, but are not limited to, starches; celluloses and derivatives thereof, e.g., microcrystalline cellulose, e.g., AVICEL PH from FMC (Philadelphia, Pa.), hydroxypropyl cellulose hydroxylethyl cellulose and hydroxylpropylmethyl cellulose METHOCEL from Dow Chemical Corp. (Midland, Mich.); sucrose; dextrose; corn syrup; polysaccharides; and gelatin. The binder, e.g., may be present in an amount from about 1% to about 50%, e.g., 2-20% by weight of the composition.

Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 0% to about 40% by weight of the composition, e.g., 20-25% by weight of the composition.

Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose, glyceryl behenate, stearic acid, hydrogenated castor oil, glyceryl monostearate, and sodium stearyl fumarate. The lubricant, e.g., may be present in an amount from about 0.1% to about 5% by weight of the composition; whereas, the glidant, e.g., may be present in an amount from about 0.1% to about 10% by weight.

The next step is wet massing the powder blend by adding a granulation liquid while agitating, or kneading, the powder blend until the powder blend is wetted with the granulation liquid to form a wet mass. For example, 10-30% (w/w) granulation liquid is added to the powder blend. Alternatively, 10-25% (w/w), e.g., 20-25%, granulation liquid can be added to the powder blend. The granulation liquid, for example is pharmaceutically acceptable and volatile. Examples of suitable granulation liquids include, but are not limited to, water, organic solvents (e.g., methanol, ethanol, isopropanol, acetone) either alone or in combination. An example of a combination granulation liquid includes water, ethanol and isopropanol together.

Alternatively, the wet granulation process may begin with the therapeutic compound as a powder by itself. During wet massing, the granulation liquid that is introduced to the powder is a solvent containing a dissolved excipient, e.g., a binder. Irrespective of how wet-massing takes place, after wet-massing, a pharmaceutical composition containing the therapeutic compound and at least one pharmaceutically acceptable excipient is wetted by the granulation liquid. In one exemplary embodiment, water is used as the granulation liquid.

The wet mass is optionally sieved forming moist, or damp, granulates. The wet mass, e.g., can be sieved through a mesh, such as a 5 mm screen. One of ordinary skill in the art can select the appropriate size of the screen in order to from the most appropriate granulate size.

In an alternative embodiment, a comminuting mill can be used in lieu of the screen or sieve. Examples of a comminuting mill include, but are not limited to, a Stokes oscillator, a Colton rotary granulator, a Fitzpatrick comminuting mill, a Stokes tornado mill.

In yet another alternative embodiment, a high-speed mixer equipped with, e.g., a chopper blade, can be used to replace either the screen or the comminuting mill. This, e.g., allows the wet massing and granulating to be combined into a single step.

The moist granulates, for example, are subsequently dried. For example, the moist granulates can be collected on trays and transferred to a drying oven. Alternatively, the moist granulates can be placed in a drying cabinet with circulating air current and thermostatic heat control. Yet another option is to dry the moist granulates in a fluidized-bed drier. In this exemplary embodiment, the moist granulates are suspended and agitated in a warm air stream such that the moist granulates are maintained in motion. For example, the temperature can be from about room temperature to about 90° C., e.g., 70° C. The moist granulates are dried to a loss on drying (“LOD”) value less than or equal to about 2%, e.g., less than 1%; e.g., 0.5-1%, by weight of the composition. Drying can take place within or apart from the pharmaceutical granulation equipment.

Subsequent to drying, the granulates can be further sieved, i.e., dry screened, alone or in combination with at least one excipient. This typically results in a more uniform particle size of the granulate, preparing the granulates for further processing into a solid oral dosage form.

The granulates may be formulated with excipients into oral forms, e.g., solid oral dosage forms, such as tablets, pills, lozenges, caplets, capsules or sachets. To make, e.g., a tablet, the granulates are combined or blended with at least one excipient, e.g., a lubricant, to form a mixture. The blending can be accomplished using any conventional pharmaceutical equipment, e.g., a V-blender.

Furthermore, any additional excipients used can be sieved separately from the granulates or concurrently with the sieving of the granulates as described in the aforementioned dry sieving step. One of ordinary skill in the art will appreciate the necessary particle size of each component that is necessary for the particular pharmaceutical, composition being formulated. For example, suitable particle sizes, include those of less than equal to 1,000 μm, 750 μm, 500 μm or 250 μm.

The blended mixture can, e.g., be subsequently compacted into a tablet (e.g., by using a tablet press) or encapsulated into a capsule. The solid oral dosage forms may be subject to further conventional processing as known to one of ordinary skill in the art, e.g., imprinting, embossing or coating.

The present invention provides the use of a composition according to the present invention comprising a therapeutic compound in the manufacture of a medicament for the treatment and/or prevention of conditions related to Flaviviridae infections.

The following examples are illustrative, but do not serve to limit the scope of the invention described herein. The examples are meant only to suggest a method of practicing the present invention.

Quantities of ingredients, represented by percentage by weight of the pharmaceutical composition, used in each example are set forth in the respective tables located after the respective descriptions.

### EXAMPLE 1

### Tablets Containing Compound I Prepared by Wet Granulation

Compound I, the dihydrochloride salt of val-mCyd, has a high density and a compact particle shape, making the therapeutic compound less ideal for dry granulation processing. Furthermore, the high drug load in the exemplary tablets render the therapeutic compound less appropriate for dry granulation processing. Alternatively, wet granulation is used as a processing method for Compound I.

Compound I having a particle size distribution, d50 of about 14-71 microns is combined with two types of cellulose: microcrystalline cellulose available as AVICEL PH 101 from FMC Corp. (Philadelphia, Pa.) and hypromellose available as CELLULOSE HP-M 603 from Shin Etsu (New York, N.Y.). The ingredients are combined in a Collette Gral granulator. Blending of the ingredients, e.g., is implemented with the impeller velocity at setting 1 (e.g., approximately 203 rpm) and the chopper at setting 1 (approximately 1,500 rpm).

Once blended, a granulation liquid, deionized water, is added for wet kneading. The impeller and chopper speeds are increased to setting 2 (e.g., 306 rpm and 500 rpm, respectively, for the impeller and chopper). The kneading is conducted at room temperature.

After granulation, the moist granulates are dried in a fluid-bed dryer with an air inlet temperature of approximately 70° C. to obtain granulates. The moist granulates are dried such that there is a LOD value below 1.0%. The granulates are subsequently sieved using a screen with a mesh size of 1.25 mm or 0.8 mm.

Separately, additional excipients: cellulose, coarse quality cellulose commercially-available as CELLULOSE MK-GR from Rettenmaier & Söhne GmbH (Weissenborn, Germany), cross-linked polyvinylpyrrolidone commercially available as PLASDONE from ISP Corporation (Wayne, N.J.), silicon dioxide available as AEROSIL 200 from Degussa (Parsippany, N.J.) and magnesium stearate are combined and sieved with a screen that has a mesh size of 1.0 mm. The additional excipients are mixed with the granulates. The mixture is then compressed into individual tablets yielding a tablet weight of 700 mg for a dosage strength of 400 mg of Compound I.

Table 1 shows the formulations of Example 1.

| | Amount | Percentage |
|---|---|---|
| | per tablet | per tablet |
| Ingredients | (mg) | (w/w %) |
| Compound I | 482.0 | 68.9 |
| Microcrystalline cellulose | 114.2 | 16.3 |
| Hypromellose | 14.0 | 2.0 |
| Microcrystalline cellulose, granular | 42.9 | 6.1 |
| Crospovidone | 35.0 | 5.0 |
| Silicon dioxide | 3.5 | 0.5 |
| Magnesium stearate | 8.4 | 1.2 |
| Total (core) | 700.0 | 100.0 |
| Film coat (e.g., of pale orange brown color) | 721.0 | — |

As mentioned above, Compound I is a salt of an ester which is potentially sensitive to hydrolysis in the presence of water. Surprisingly, the addition of the granulation liquid of deionized water does not result in an increase in degradation, e.g., due to hydrolysis, after wet granulation as long as the LOD value is less than or equal to about 1% for the dried granulate.

It is understood that while the present invention has been described in conjunction with the detailed description thereof that the foregoing description is intended to illustrate and not limit the scope of the invention, which is defined by the scope of the following claims. Other aspects, advantages and modifications are within the scope of the claims.

What is claimed:

1. A method for preparing a pharmaceutical composition comprising the steps of:
(a) forming a powder blend of val-mCyd or a pharmaceutically acceptable salt, ester, prodrug or derivative thereof, and at least one pharmaceutically acceptable excipient;
(b) wet massing and kneading the powder blend with a granulation liquid to form moist granulates; and
(c) drying the moist granulates to form granulates.

2. The method of claim 1, further comprising processing the granulates into a solid oral dosage form.

3. The method of claim 1, wherein the val-mCyd is the dihydrochloride salt thereof.

4. The method of claim 1, wherein the granulation liquid comprises water.

5. The method of claim 4, wherein the granulation liquid is present in a concentration from about 10% to about 30% by weight of the powder blend.

6. The method of claim 1, further comprising the step of sieving the granulates.

7. The method of claim 1, wherein the moist granulates are dried to a loss on drying (“LOD”) value less than or equal to about 2% by weight of the moist granulates after drying.

8. The method of claim 7, wherein the drying value is less than or equal to about 1% by weight of the moist granulates after drying.

9. The method of claim 1, wherein the drying takes place at a temperature from room temperature to 90° C.

10. A method for preparing a pharmaceutical composition comprising the steps of:
(a) forming a powder blend of val-mCyd or a pharmaceutically acceptable salt, ester, prodrug or derivative thereof, and at least one pharmaceutically acceptable excipient;
(b) adding a granulation liquid to the powder blend under agitation to form a wet mass;
(c) granulating the wet mass to form moist granulates; and
(d) drying the moist granulates to form granulates.

11. The method of claim 10, further comprising processing the granulates into a solid oral dosage form.

12. The method of claim 10, wherein the val-mCyd is the dihydrochloride salt thereof.

13. The method of claim 10, wherein the granulation liquid comprises water.

14. The method of claim 13, wherein the granulation liquid is present in a concentration from about 10% to about 30% by weight of the powder blend.

15. The method of claim 10, further comprising the step of sieving the granulates.

16. The method of claim 10, wherein the moist granulates are dried to a LOD value less than or equal to about 2% by weight of the moist granulates.

17. The method of claim 16, wherein the drying value is less than or equal to about 1% by weight of the moist granulates after drying.

18. The method of claim 10, wherein the drying takes place at a temperature from room temperature to 90° C.

19. A product produced by the process of claim 1.

20. A pharmaceutical composition comprising, val-mCyd or a pharmaceutically acceptable salt, ester, prodrug or derivative thereof, and at least one pharmaceutically acceptable excipient, wherein said pharmaceutical composition is a solid oral dosage form.
